(19) United States
(12) Patent Application Publication
(10) Pub. No.: US 2018/0275117 A1
(43) Pub. Date: Sep. 27, 2018

(54) MATERIALS AND METHODS TO IMPROVE ACCURACY OF ASSAYS

(71) Applicant: UNIVERSITY OF FLORIDA RESEARCH FOUNDATION, INC., GAINESVILLE, FL (US)

(72) Inventor: DANIEL J. GIBSON, Gainesville, FL (US)

(21) Appl. No.: 15/549,168

(22) PCT Filed: Feb. 18, 2016
(86) PCT No.: PCT/US2016/018375

§ 371 Date: Aug. 6, 2017

Related U.S. Application Data
(60) Provisional application No. 62/117,468, filed on Feb. 18, 2015.

(51) Int. Cl. G01N 33/52; C12Q 1/37; G01N 33/84; G01N 33/96

(52) U.S. Cl. CPC G01N 33/52; G01N 33/96; G01N 33/84; C12Q 1/37

(57) ABSTRACT

The current invention provides a method of calculating the volume of a sample mixed into a known volume of an assay reaction mixture wherein the assay reaction mixture comprises a solute at a known concentration, wherein the solute is non-reactive with other constituents of the assay reaction mixture, is non-reactive with the constituents of the sample, is absent in the sample, and does not participate in the assay reaction. The current invention also pertains to assay reaction mixtures used in the methods of the current invention.

### CROSS-REFERENCE TO A RELATED APPLICATION

This application claims the benefit of U.S. Provisional Application Ser. No. 62/117,468, filed Feb. 18, 2015, which is incorporated herein by reference in its entirety.

### BACKGROUND OF INVENTION

Analytical assays, for example, biological assays (bioassays), are sensitive to variations in the sample volume put into the assay reaction. Many medically relevant assays use imprecise methods of collecting biofluid samples. This imprecision paired with the sensitivity to variations in input volume lead to errors in biological assays.

### BRIEF SUMMARY

The current invention provides assay reaction mixtures and methods to accurately measure the volume of a sample used in an assay, particularly, a bioassay. Accordingly, the current invention provides an assay reaction mixture for detection and/or quantification of a molecule in a biofluid sample obtained from a subject, wherein the assay reaction mixture comprises a solute that is non-reactive with other constituents of the assay reaction mixture, non-reactive with the constituents of the biofluid sample, absent in the biofluid sample, and does not participate in the assay reaction.

In some embodiments, the solute can be a pigment or a fluorophore and therefore, can be measured spectroscopically by, for example, absorption spectroscopy or fluorescent spectroscopy.

The current invention also provides a method for calculating the volume of a biofluid sample mixed with a known volume of an assay reaction mixture for detection and/or quantification of a molecule in the biofluid sample, wherein the assay reaction mixture comprises a solute at a known concentration, and wherein the solute is non-reactive with other constituents of the assay reaction mixture, non-reactive with the constituents of the biofluid sample, absent in the biofluid sample, and does not participate in the assay reaction, the method comprising:

a) measuring the concentration of the solute in the mixture produced after the biofluid sample is mixed with the assay reaction mixture, and

b) calculating the volume of the biofluid sample based on the reduction in the concentration of the solute in the mixture of the biofluid sample and the assay reaction mixture compared to the concentration of the solute in the assay reaction mixture.

### BRIEF DESCRIPTION OF DRAWINGS

FIG. 1 shows mixing of an assay reaction mixture with a biofluid sample producing dilution of the solute present in the assay reaction mixture.

FIG. 2 shows examples of biofluid samples of insufficient volumes mixed with an assay reaction mixture.

FIG. 3 shows an example of a biofluid sample of excessive volume mixed with an assay reaction mixture.

FIG. 4 shows an example of biofluid sample mixed with an assay reaction mixture, wherein the biofluid sample has a volume that is nominally different from the desired input volume.

FIG. 5 shows an example of corrected standard curve adjusted based on the volume of the standard solution added in the assay reaction mixture.

### DETAILED DISCLOSURE

The current invention provides materials and methods for detection and/or quantification of a molecule in a sample. In one embodiment the assay reaction mixture comprises a solute, wherein the solute is non-reactive with other constituents of the assay reaction mixture, is non-reactive with the constituents of the sample, is absent in the sample, and does not participate in the assay reaction.

A key concern for point of care diagnostics is assay performance/accuracy, which can be adversely affected due to variations in sample collection and introduction into the assay mixture. The current invention provides improved clinical diagnostic assays. In a specific embodiment, the assay is improved by including a solute, for example, a pigment, in the assay reaction mixture. The solute added to the assay reaction mixture can be monitored for degree of dilution subsequent to mixing with the sample and the sample volume can then be measured based on the degree of dilution of the solute.

For the purposes of the current invention, “a solute non-reactive with other constituents of the assay reaction mixture and non-reactive with the constituents of the sample” means that the solute does not significantly chemically or physically interact with the constituents of the assay reaction mixture or the constituents of the sample. Hence, the amount of the solute present in the assay reaction mixture before mixing with the sample remains the same after the assay reaction mixture is mixed with the sample.

In addition, “the solute does not participate in the assay reaction” means that the solute is not significantly involved in the chemical reactions that produce measurable outcomes used in detection and/or quantification of an analyte in the sample. Also, the solute is not produced during the chemical reactions that occur in an analysis of the analyte. Thus, the solute is inert to the constituents of the assay reaction mixture and the sample. Given the guidance provided herein, a person of ordinary skill in the art can select an appropriate solute for a particular assay based on the reactants involved in the assay.

In one embodiment of the invention, the solute is a pigment or a fluorophore and, accordingly, the concentration of the solute is measurable spectroscopically.

For the purposes of the current invention the term “pigment” refers to a compound having specific absorption characteristics, e.g., absorption within the ultraviolet, visible, or the infrared spectrum of light. The pigment can be quantified based on the absorption of the solution at a particular wavelength.

In one embodiment, the solute is a pigment and can be measured by absorption spectroscopy. Absorption spectroscopy can be performed in ultra-violet, visible, or infra-red spectrum.

In another example, the solute is a fluorophore and can be measured by fluorescent spectroscopy.

For the purposes of the current invention, a fluorophore is a fluorescent chemical compound that can re-emit light upon light excitation. Usually, the emitted light has a different wavelength than that of the excitation light. Various fluorophores that can be used in the compositions and methods of the current invention are well known to a person of ordinary skill in the art and such embodiments are within the purview of the current invention.

The assay reaction mixtures of the current invention can be used to accurately determine the volume of sample mixed with the assay reaction mixture. Accordingly, the current invention provides methods of calculating the volume of sample mixed into a known volume of an assay reaction mixture wherein the assay reaction mixture comprises a solute at a known concentration, wherein the solute is non-reactive with other constituents of the assay reaction mixture, is non-reactive with the constituents of the sample, is absent in the sample, and does not participate in the assay reaction, the method comprising:

a) measuring the concentration of the solute in the mixture produced after the sample is mixed with the assay reaction mixture, and

b) calculating the volume of the sample based on the reduction in the concentration of the solute in the mixture of the sample and the assay reaction mixture compared to the concentration of the solute in the assay reaction mixture.

In preferred embodiments, the sample is a biofluid sample and the assay is a bioassay, particularly, a bioassay to detect and/or quantify a clinically relevant biomolecule.

In accordance with the subject invention, an assay can be rejected based on insufficient dilution, i.e., insufficient sample, or based on excessive dilution, i.e., too much sample. The dilutions of the solute within an acceptable range based on predetermined boundaries can be used to calculate a scaling factor that facilitates accurate measurement of the sample volume that nominally deviates from the desired input volume. A person of ordinary skill in the art can modify the assay reaction mixture of the current invention to suit various types of assays and such embodiments are within the purview of the current invention.

Because the methods of the current invention can be used in clinically-relevant bioassays, the samples used in the methods of the current invention include biofluids obtained from a subject. Non-limiting examples of biofluids that can be used in the methods of the current invention include exhaled breath, whole blood, blood plasma, blood serum, urine, tears, semen, saliva, buccal mucosa, interstitial fluid, lymph fluid, meningeal fluid, amniotic fluid, glandular fluid, sputum, feces, perspiration, mucous, vaginal secretion, cerebrospinal fluid, wound exudate, wound homogenate, wound fluid, aqueous humor, vitreous humor, bile, endolymph, perilymph, pericardial fluid, pleural fluid, and synovial fluid. The biofluids can be appropriately treated before they are used pursuant to the methods of the current invention.

Biofluids as used in the current invention also include extracts of a tissue. The tissues can be appropriately treated to produce biofluid for use according to the current invention. A person of ordinary skill in the art can utilize various tissue treatments to produce biofluids.

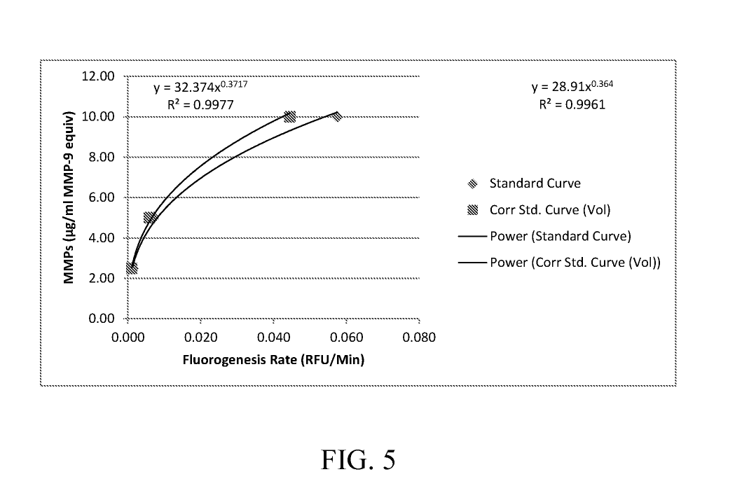

In a further embodiment of the invention, the sample is a standard solution containing a known concentration of the analyte to be assayed. Such standard solutions are used to produce a standard curve of the analyte. The assay reaction mixture of the current invention allows accurate determination of the standard solution mixed in the set of reactions used to produce the standard curve for the analyte. An example of a standard curve and the corrected standard curve is provided in FIG. 5.

The current invention further provides assay kits for detection and/or quantification of an analyte in a sample. The assay kit of the current invention can comprise an assay reaction mixture comprising a solute at a known concentration, wherein the solute is non-reactive with other constituents of the assay reaction mixture, is non-reactive with the constituents of the sample, is absent in the sample, and does not participate in the assay reaction. The kit can separately provide various constituents of the assay reaction mixture. A user can then mix the contents to produce the assay reaction mixture for use according to the methods of the current invention.

In certain embodiment of the current invention, the assay kits are designed for detection and/or quantification of a clinically relevant biomolecule in a biological sample such as, for example, a biofluid sample, obtained from a subject. Non-limiting examples of the biomolecules that can be assayed according to the kits and methods of the current invention include matrix metalloproteinase (MMP), neutrophil elastase (NE), and nitrogen dioxide (NO₂). In certain embodiments, the assay is Fluorescence Resonance Energy Transfer (FRET)-based assay such as, for example, MMP-FRET, human NE-FRET, or NO₂ fluorescence assay.

### EXAMPLES

Following are examples that illustrate embodiments and procedures for practicing the invention. These examples should not be construed as limiting.

### Example 1—Determining the Volume of the Sample and Rejecting/Accepting a Test Result

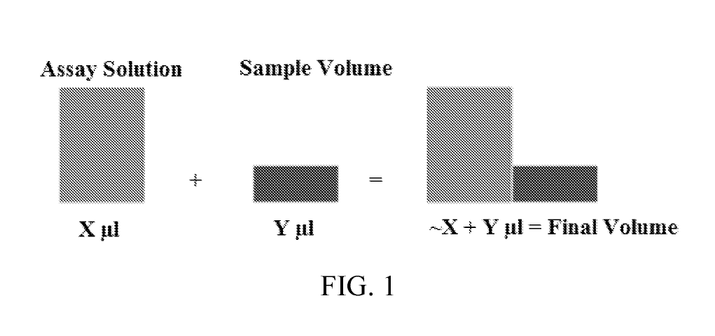

In an assay, 350 μl of assay reaction mixture is mixed with 150 μl of sample to produce 30% v/v dilution of the chemicals, including a solute, within the assay reaction mixture (FIG. 1). The solute is a pigment or a fluorophore that can be measured spectroscopically. The pigment concentration can be measured either by absorption spectroscopy or by fluorescence spectroscopy and the pigment or the fluorophores can be quantified to determine how the anticipated dilution deviates from 30%.

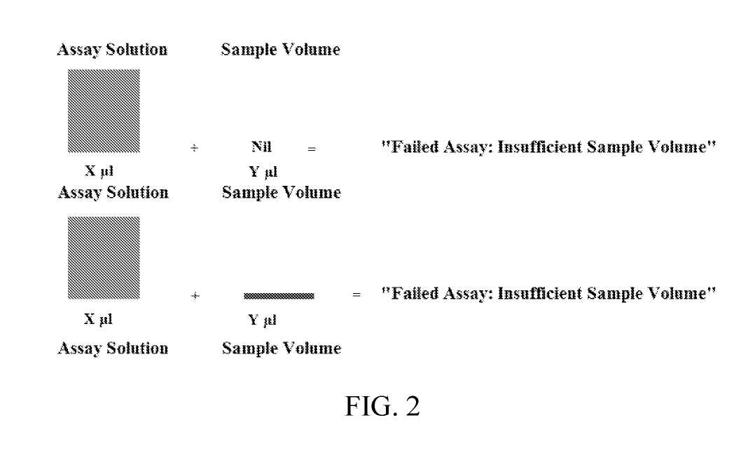

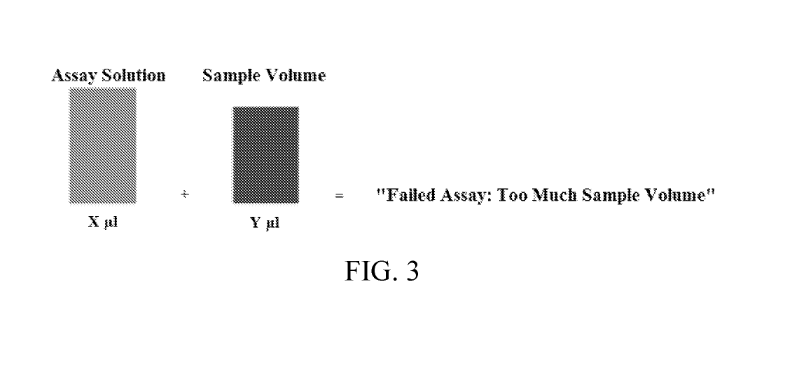

If there is no dilution, or the dilution is below the nominal 30% dilution by a predetermined level, for example, 10-20% below nominal, then the assay can be rejected as invalid due to insufficient sample volume (FIG. 2). Similarly, if the dilution is above the nominal 30% dilution by a predetermined level, for example, 10-20% above nominal, then the assay can be rejected as invalid due to excessive sample volume (FIG. 3).

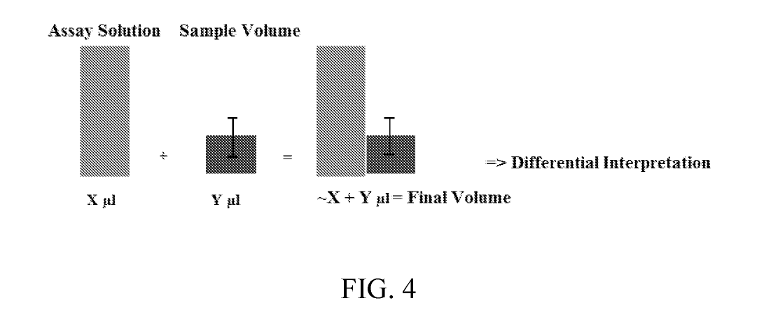

In the event that the dilution is nominally above or below the nominal 30% dilution, e.g., the dilution is within a predetermined dilution range, for example, within ±10-20%, the dilution can be used to establish a scaling factor (FIG. 4). The scaling factor can then be applied to the assay such that a different set of calibration coefficients are used for every sample. Also if the dilution is within the linear range of the assay, a simple scaling of the data can be used to improve the accuracy of the assay.

### Example 2—Determining the Volume of Biofluid Samples in Assays for Clinically Relevant Biomolecules

The compositions and methods of the current invention can be used to determine the volume of biofluid samples used in assays for clinically relevant biomolecules, for example, in medical diagnostic assays. Non-limiting examples of such assays include the assays for matrix MMP-FRET assay, NE-FRET assay, or NO₂-FRET assay.

In further embodiments, the current invention is modified to suit bioassays described in, for example, United States Patent Application Publication Nos. 2012/0136054, 2012/0135443, 2012/0122133, 2012/0078162, 2009/0258382, and 2008/0176263, the contents of which are incorporated herein by reference in their entireties.

I claim:

1. An assay reaction mixture for detection and/or quantification of a molecule in a sample, the assay reaction mixture comprising a solute, wherein the solute is non-reactive with other constituents of the assay reaction mixture, is non-reactive with the constituents of the sample, is absent from the sample, and does not participate in the assay reaction.

2. The assay reaction mixture of claim 1, wherein the concentration of the solute is measurable spectroscopically.

3. The assay reaction mixture of claim 2, wherein the solute is a pigment or a fluorophore.

4. The assay reaction mixture of claim 1, wherein the sample is a biofluid sample obtained from a subject and the molecule is a clinically relevant biomolecule.

5. The reaction mixture of claim 4, wherein the biofluid sample is exhaled breath, whole blood, blood plasma, blood serum, urine, tears, semen, saliva, buccal mucosa, interstitial fluid, lymph fluid, meningeal fluid, amniotic fluid, glandular fluid, sputum, feces, perspiration, mucous, vaginal secretion, cerebrospinal fluid, wound exudate, wound homogenate, wound fluid, aqueous humor, vitreous humor, bile, endolymph, perilymph, pericardial fluid, pleural fluid, or synovial fluid.

6. The reaction mixture of claim 4, wherein the biofluid sample is an extract of a tissue.

7. The reaction mixture of claim 4, wherein the biomolecule is matrix metalloproteinase (MMP), neutrophil elastase (NE), or nitric oxide (NO₂).

8. A method of calculating the volume of a sample mixed into a known volume of an assay reaction mixture wherein the assay reaction mixture comprises a solute at a known concentration, wherein the solute is non-reactive with other constituents of the assay reaction mixture, is non-reactive with the constituents of the sample, is absent in the sample, and does not participate in the assay reaction, the method comprising:
a) measuring the concentration of the solute in the mixture produced after the sample is mixed with the assay reaction mixture, and
b) calculating the volume of the sample based on the reduction in the concentration of the solute in the mixture of the sample and the assay reaction mixture compared to the concentration of the solute in the assay reaction mixture.

9. The method of claim 8, wherein the sample is a biofluid sample from a subject and the molecule is a clinically relevant biomolecule.

10. The method of claim 8, wherein the concentration of the solute is measured spectroscopically.

11. The method of claim 10, wherein the solute is a pigment or a fluorophore.

12. The method of claim 9, wherein the biofluid sample is exhaled breath, whole blood, blood plasma, blood serum, urine, tears, semen, saliva, buccal mucosa, interstitial fluid, lymph fluid, meningeal fluid, amniotic fluid, glandular fluid, sputum, feces, perspiration, mucous, vaginal secretion, cerebrospinal fluid, wound exudate, wound homogenate, wound fluid, aqueous humor, vitreous humor, bile, endolymph, perilymph, pericardial fluid, pleural fluid, or synovial fluid.

13. The method of claim 9, wherein the biofluid sample is an extract of a tissue selected from brain, eyes, pineal gland, pituitary gland, thyroid gland, parathyroid glands, thorax, heart, lungs, esophagus, thymus gland, pleura, adrenal glands, appendix, gall bladder, urinary bladder, large intestine, small intestine, kidneys, liver, pancreas, spleen, stoma, prostate gland, testes, ovaries, or uterus.

14. The method of claim 9, wherein the biomolecule is MMP, NE, or NO₂.

15. An assay kit for estimation of a molecule in a sample, the assay kit comprising an assay reaction mixture comprising a solute at a known concentration, wherein the solute is non-reactive with other constituents of the assay reaction mixture, is non-reactive with the constituents of the sample, is absent in the sample, and does not participate in the assay reaction.

16. The assay kit of claim 15, wherein the kit is designed for estimation of a clinically relevant biomolecule in a biofluid sample obtained from a subject.

17. The assay kit of claim 15, wherein the clinically relevant biomolecule is MMP, NE, or NO₂.
